(19) United States
(12) Patent Application Publication
(10) Pub. No.: US 2025/0271443 A1
(43) Pub. Date: Aug. 28, 2025

(54) SOLUBLE SINGLE-CHAIN DIMERS FROM CLEAVABLE SINGLE CHAIN TRIMERS

(71) Applicants: Institute for Systems Biology, Seattle, WA (US); California Institute of Technology, Pasadena, CA (US)

(72) Inventors: William Chour, Los Angeles, CA (US); James R. Heath, Seattle, WA (US); Jingyi Xie, Seattle, WA (US)

(73) Assignees: Institute for Systems Biology, Seattle, WA (US); California Institute of Technology, Pasadena, CA (US)

(21) Appl. No.: 18/857,944

(22) PCT Filed: Apr. 20, 2023
(86) PCT No.: PCT/US2023/066002

§ 371 Date: Oct. 18, 2024

Related U.S. Application Data
(60) Provisional application No. 63/333,529, filed on Apr. 21, 2022.

(51) Int. Cl. G01N 33/68; C07K 14/74

(52) U.S. Cl. CPC G01N 33/6842; C07K 14/70539; C07K 2319/50

(57) ABSTRACT

The present disclosure describes, in part, soluble single-chain dimers (sSCDs) generated from cleavable single-chain trimers (cSCTs), compositions and methods for their production, as well as applications thereof related to characterization of antigen-specific CD8+ T cells and treatments

### CROSS REFERENCE TO RELATED APPLICATIONS

This application claims priority to U.S. Provisional Application No. 63/333,529, filed Apr. 21, 2022, which is incorporated by reference in its entirety.

### ACKNOWLEDGMENT OF GOVERNMENT SUPPORT

This invention was made with government support under grant number 1 R01 CA264090-01 awarded by the National Institutes of Health and grant number HHSO10201600031C awarded by the Biomedical Advanced Research and Development Authority. The government has certain rights in the invention.

### FIELD

This disclosure relates to soluble single-chain dimers (sSCDs) generated from cleavable single-chain trimers (cSCTs), compositions and methods for their production and use.

### BACKGROUND

Saini et al. in 2019 reported the development of a peptide-free pMHC construct (Saini et al. “Empty peptide-receptive MHC class I molecules for efficient detection of antigen-specific T cells. Science Immunology, 4 (37), July 2019. ISSN 2470-9468. doi: 10.1126/sciimmunol. aau9039; available at immunology.sciencemag.org/lookup/doi/10.1126/sciimmunol.aau9039). Typically, MHCs can only be stabilized and folded together in the presence of a peptide; otherwise, the two subunits will not stably bind together. This requirement has posed a challenge for applications such as high-throughput pMHC production, which have traditionally necessitated individual three component refolding, and more recently have moved onto UV exchange as the main standard for library generation. By introducing two HLA mutations (Y84C and A139C), the authors were able to refold bacterially expressed MHC in the absence of a peptide, therefore generating peptide-free stabilized MHC constructs. They proceeded to demonstrate that these reagents can essentially be used as off-the-shelf reagents, in which the user can mix the empty MHCs with any peptide desired to generate functional pMHCs. While peptides of interest were loaded in a separate step by UV-mediated or temperature-induced peptide exchange or via disulfide-stabilized empty MHC-I molecules (for HLA-A*02:01, HLA-A*24:02, and H-2K^{b}), the β2M and HLA components were bacterial expressed and refolded with dipeptides before purification, and with limited reagent stability (Saini et al., supra).

This alternative approach to generate high-throughput libraries was applied in their concomitant paper, in which they analyzed binding affinities of an alanine scan library of the SIINFEKL (SEQ ID NO: 1) peptide, demonstrating the potential of their dual mutation system (Moritz et al. “High-throughput peptide-MHC complex generation and kinetic screenings of TCRs with peptide-receptive HLA-A*02:01 molecules. Science Immunology, 4 (37):eaav0860, July 2019. ISSN 2470-9468. doi: 10.1126/sciimmunol. aav0860; available at immunology.sciencemag.org/lookup/doi/10.1126/sciimmunol.aav0860).

### SUMMARY

Provided herein are major histocompatibility complex (MHC) Class I cleavable single chain trimer (cSCT) proteins, soluble single-chain dimer (sSCD) proteins generated therefrom, and assays that can be used for, or that benefit from, discovery or characterization of T cells and T cell receptors (TCRs).

Provided herein are major histocompatibility complex (MHC) Class I cleavable single chain trimer (cSCT) proteins. The cSCT proteins include covalently linked subunits in the following N-terminal to C-terminal order a peptide, a unique protease cleavage site, a β2 microglobulin (β2m) protein, and a human leukocyte antigen (HLA) protein. In some examples, the unique protease cleavage site is comprised as a linker joining the peptide to the β2m protein. In additional examples, the MHC Class I cSCT protein includes covalently linked components in the following N-terminal to C-terminal order: the peptide; the unique protease cleavage site-β2m linker (L1); the β2m protein; a β2m-HLA linker (L2); the HLA protein; and optionally, one or more purification tags. In some implementations, the unique protease cleavage site-β2m linker L1 includes one or more Gly-Gly-Gly-Gly-Ser (GGGGS; SEQ ID NO: 1) amino acid repeats (such as 1, 2, 3, or more GGGGS (SEQ ID NO: 1) repeats and the unique protease cleavage site is internal to, or flanking, the one or more GGGGS (SEQ ID NO: 1) amino acid repeats.

In some implementations the unique protease cleavage site is a tobacco etch virus (TEV) protease cleavage site. In some examples, the TEV protease cleavage site includes the amino acid sequence Glu-Asn-Leu-Tyr-Phe-Gln-Gly/Ser (ENLYFQG/S; SEQ ID NO: 2) and the TEV protease cleaves between the Gln and Gly/Ser residues. In certain examples, the unique protease cleavage site-β2m linker L1 is selected from: ENLYFQGGGGSGGGGGGGGS (SEQ ID NO: 3); GGGGSENLYFQGGGGSGGGGS (SEQ ID NO: 4); GGGGSGGGGSENLYFQGGGGS (SEQ ID NO: 5); and GGGGSGGGGSGGGGSENLYFQG/S (SEQ ID NO: 6).

In some implementations the MHC Class I cSCT protein includes one or more purification tags. In some examples, the one or more purification tags are selected from a peptide that can be biotinylated and a polyhistidine peptide.

In other implementations, the HLA protein subunit of the MHC Class I cSCT protein includes one or more amino acid substitutions selected from the group consisting of H74L, Y84C, Y84A, A139C, D227K, T228A, and A245V.

In some implementations the peptide subunit of the MHC Class I cSCT protein is an antigen peptide, a self peptide, or a placeholder peptide. In some examples, the antigen peptide is selected from a tumor-associated peptide, a neoantigen peptide, an autoimmune peptide, a fungal peptide, a bacterial peptide, and a viral peptide.

Also provided are nucleic acids encoding the disclosed MHC Class I cSCT proteins and vectors including the nucleic acids. Cells transformed with the disclosed nucleic acids or vectors are also provided.

Provided herein are major histocompatibility complex (MHC) Class I soluble single chain dimer (sSCD) proteins produced by cleavage of the disclosed cSCT proteins at the unique protease cleavage site. In some examples, the Class I sSCD is free of the peptide. In further examples, the MHC Class I sSCD is reconstituted with a peptide selected from an antigen peptide, a self peptide, or a placeholder peptide. In some implementations, the sSCD protein and peptide are assembled as a stable multimer, such as a tetramer. In some examples, the stable multimer is attached to a surface through a purification tag.

Also provided are major histocompatibility complex (MHC) Class I soluble single chain dimer (sSCD) proteins, wherein the sSCD protein includes covalently linked first and second subunits in the N-terminal to C-terminal direction: a β2 microglobulin (β2m) protein subunit and a human leukocyte antigen (HLA) protein subunit. In some implementations, the sSCD protein includes a third subunit that is non-covalently bound by the β2m protein and HLA protein subunits, the third subunit including a peptide selected from an antigen peptide, a self peptide, or a placeholder peptide. In some examples, the antigen peptide is selected from a tumor-associated peptide, a neoantigen peptide, an autoimmune peptide, a fungal peptide, a bacterial peptide, and a viral peptide. In some examples, the sSCD protein is stable in aqueous solution.

In additional implementations, the HLA protein of the MHC Class I sSCD protein includes one or more amino acid substitutions selected from the group consisting of H74L, Y84C, Y84A, A139C, D227K, T228A, and A245V. In other implementations, the MHC Class I sSCD protein further includes one or more purification tags.

Also provided is a library including a plurality of the disclosed MHC Class I sSCD proteins.

Provided herein are methods of identifying an antigen-specific CD8+ T cell. The methods include contacting a T cell population with one or more of the stable multimers of a disclosed MHC Class I sSCD protein; and identifying a CD8+ T cell reactive thereto.

The foregoing and other features of the disclosure will become more apparent from the following detailed description, which proceeds with reference to the accompanying figures.

### BRIEF DESCRIPTIONOF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more implementations as provided herein.

FIGS. 1A-1C: SCT design for MHC Class I pMHC constructs. FIG. 1A. SCTs encoding Class I pMHC molecules are constructed by Gibson assembly from two fragments, enabling modular insertion of any desired Class I HLA subunit to design a template plasmid for peptide insertion. FIG. 1B. Template SCT constructs are ligated into pcDNA3.1 vector by restriction digest and ligation. FIG. 1C. An SCT library containing various peptide elements can be constructed from an initial template plasmid by inverse PCR and ligation.

FIG. 2: Illustration of the general location of unique protease cleavage site in linker L1 of the construct depicted in FIGS. 1A-1C.

FIG. 3: Expression of cleavable SCTs. A*02:01 SCT plasmids (template D9) loaded with MART-1 peptide (ELAGILGILTV; SEQ ID NO: 12) were modified with a TEV protease cleavage site insertion within the L1 linker. 1=SEQ ID NO: 13; 2=SEQ ID NO: 14, 3=SEQ ID NO: 15, 4=SEQ ID NO: 16, 5=SEQ ID NO: 17. Shown above is a reduced SDS-PAGE of transfection results from various designs.

FIG. 4: SCTs may be purified at larger scale to obtain milligram-scale yields. A*02:01 SCT plasmid (D3 template) loaded with the WT1 peptide (RMFPNAPYL; SEQ ID NO: 19) was transfected at large scale (30 ml) for four days. The secreted protein was collected and purified by size-exclusion FPLC. Shown above is the absorbance analysis of eluent, where the desired purified SCT (column fraction A/5) appears as a singular peak.

FIG. 5: Schematic illustration of pMHC multimer preparation using peptide-receptive SCD reagents. SCT=SEQ ID NO: 10; SCD=SEQ ID NO: 11.

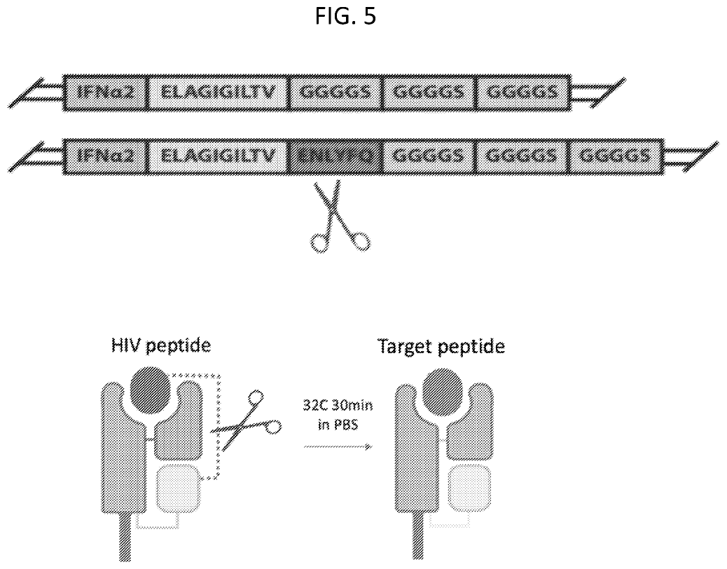

FIG. 6: Flow cytometry result for staining HLA-A*11:01 KRAS G12V-specific T cells using SCD, SCT, or other commercial pMHCs. G12V (8-16) peptide=SEQ ID NO: 7; G12V (7-16) peptide=SEQ ID NO: 8; G12D (7-16) peptide=SEQ ID NO: 9.

FIG. 7: Flow cytometry result for stability test of peptide-receptive SCD monomer or peptide-loaded SCD tetramers after two week storage. Top, SCD template was treated with TEV protease, stored in PBS at 4° C. for two weeks, then incubated with G12V (8-16) peptide, incubated with tetramer-PE, then stained. Bottom, SCD template was treated with TEV protease, immediately incubated with G12V (8-16) peptide, incubated with tetramer-PE, stored at 4° C. for two weeks before staining cells.

FIG. 8: Stability test of peptide-receptive SCD monomer after one month and three months.

### SEQUENCE LISTING

Any nucleic acid and amino acid sequences listed herein or the accompanying Sequence Listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand

SEQ ID NO: 1 is an exemplary glycine-serine
linker:
GGGGS
SEQ ID NO: 2 is an exemplary TEV cleavage site:
ENLYFQG/S
SEQ ID NOs: 3-6 are exemplary protease cleavage
site-β2m linkers:
ENLYFQGGGGSGGGGSGGGGS
(SEQ ID NO: 3)
GGGGSENLYFQGGGGSGGGGS
(SEQ ID NO: 4)
GGGGSGGGGSENLYFQGGGGS
(SEQ ID NO: 5)
GGGGSGGGGSGGGGSENLYFQG/S
(SEQ ID NO: 6)
SEQ ID NO: 7 is a G12V (8-16) peptide:
VVGAVGVGK
SEQ ID NO: 8 is a G12V(7-16) peptide:
VVVGAVGVGK
SEQ ID NO: 9 is a G12D(8-16) peptide:
VVGADGVGK
SEQ ID NO: 10 is an exemplary SCT
peptide L1 construct:
ELAGIGILTVGGGGSGGGGSGGGGS
SEQ ID NO: 11 is an exemplary SCD
peptide-cleavage site- L1construct:
ELAGIGILTVENLYFQGGGGSGGGGSGGGGS
SEQ ID NO: 12 is an exemplary MART-1 peptide:
ELAGILGILTV
SEQ ID NO: 13 is an exemplary peptide-linker
sequence:
ELAGIGILTVGGGGSGGGGSGGGGS
SEQ ID NOs: 14-17 are exemplary peptide-
protease cleavage-linker sequences:
ELAGIGILTVENLYFQGGGGSGGGGSGGGGS
(SEQ ID NO: 14)
ELAGIGILTVGGGGSENLYFQGGGGSGGGGS
(SEQ ID NO: 15)
ELAGIGILTVGGGGSGGGGSENLYFQGGGGS
(SEQ ID NO: 16)
ELAGIGILTVGGGGSGGGGSGGGGSENLYFQ
(SEQ ID NO: 17)
SEQ ID NO: 18 is an exemplary glycine-serine
(3) linker:
GGGGSGGGGSGGGGS
SEQ ID NO: 19 is an exemplary WT1 peptide:
RMFPNAPYL

### DETAILED DESCRIPTION

SCT reagents have been a powerful and versatile tool for the detection of antigen-specific T cells. However, there are some limitations for the application of SCT reagents: a) SCT template needs to be optimized for individual HLA for optimal protein expression. b) In certain cases, the SCT version of the pMHC multimer was found non-specifically binding to un-cognate TCRs (such as the A*11:01G12V SCT reagents). Such non-specific binding is not well-understood yet, but it might be reduced by HLA modification, or by the structure change due to covalently linkage between pMHC compartments. In this case, SCD reagents provide an excellent alternative for sensitive and specific detection. c) It has been known that SCT expression yield does not necessarily reflect the peptide-MHC binding affinity, which means some low affinity epitopes will generate high yield of SCT expression even when the epitope is unlikely being naturally presented by the HLA. Instead, the peptide loading process for SCD is highly dependent on the affinity between peptide and HLA, which might be beneficial for the discovery of immunogenic epitopes that is preferred for antigen presentation. SCD reagents might also be an effective tool to understand the antigen-HLA binding. d) in some studies, a list of peptides (neoantigens generated from patients, or peptides from target virus) is already being synthesized. In this case, peptide-receptive SCD reagents would beneficial since the peptides can be immediately used to prepare the pMHC tetramer reagents, reducing the cost and time of preparing SCT libraries which usually takes up to 3 weeks depending on the size of library. As disclosed herein, SCD reagents are an alternative of SCT, and are also highly scalable and stable. The preparation of peptide-loaded SCD multimer is fast, simple and cost-effective. Efficient antigen-specific T cell detection has been illustrated using peptide-receptive SCD reagents and cognate TCRs against cancer KRAS mutation. SCD tetramer demonstrated improved specificity against cognate TCRs comparing to SCT tetramer and commercial pMHC tetramer in some cases. SCD reagents can be applied for the large-scale TCR discovery for antigen-specific TCRs.

As summarized above, provided herein is a high-throughput single-chain trimer (SCT) and dimer (SCD) platform enabling production of SDTs for any pairing of peptide and Class I HLA allele. Whereas with traditional pMHC folding, epitope and HLA modularity are determined by peptide synthesis and refolded MHC subunits, respectively, the SCT and SCD platform described herein utilizes a protease cleavable SCT to generate a soluble SCD that can be loaded with a peptide of interest to determine these two variables. The facile nature of handling and scaling up these reagents enables a mix-and-match approach that allows rapid screening across a peptide library and list of HLA template variants to optimize pMHCs.

### I. MHC Class I Cleavable Single Chain Trimers

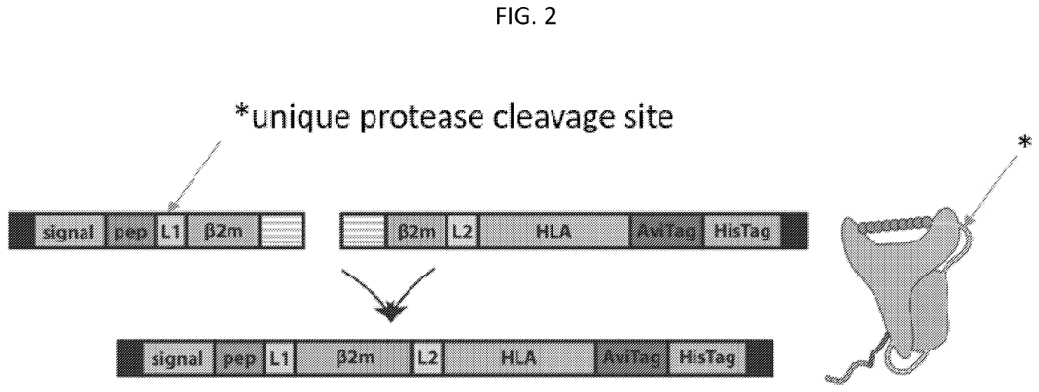

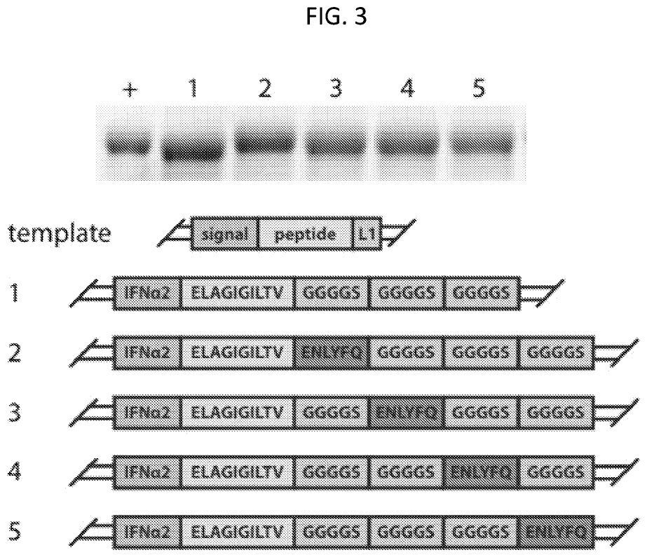

Provided herein are cSCT proteins that include as covalently linked subunits in the following N-terminal to C-terminal order a peptide, a unique protease cleavage site, a β2 microglobulin (β2m) protein, and a human leukocyte antigen (HLA) protein. In certain implementations, the unique protease cleavage site is comprised as a linker joining the peptide to the β2m protein. In particular examples, the MHC Class I cSCT protein includes covalently linked components in the following N-terminal to C-terminal order: the peptide; the unique protease cleavage site-β2m linker (L1); the β2m protein; a β2m-HLA linker (L2); the HLA protein; and optionally, one or more purification tags (see, e.g., FIG. 2). A specific aspect is where the unique protease cleavage site-β2m linker L1 comprises a Gly-Gly-Gly-Gly-Ser (GGGGS; SEQ ID NO: 1) amino acid repeat, and the unique protease cleavage site is internal to, or flanking, the GGGGS (SEQ ID NO: 1) amino acid repeat (see, e.g., FIG. 3). A specific example is where the unique protease cleavage site is a TEV protease cleavage site that recognizes the amino acid sequence Glu-Asn-Leu-Tyr-Phe-Gln-Gly/Ser (ENLYFQG/S; SEQ ID NO: 2) and cleaves between the Gln and Gly/Ser residues, and wherein the unique protease cleavage site-β2m linker L1 is selected from: ENLYFQGGGGSGGGGSGGGGS (SEQ ID NO: 3); GGGGSENLYFQGGGGSGGGGS (SEQ ID NO: 4); GGGGSGGGGSENLYFQGGGGS (SEQ ID NO: 5); and GGGGSGGGGSGGGGSENLYFQG/S (SEQ ID NO: 6). In as many implementations, the MHC Class I cSCT protein includes one or more purification tags and the one or more purification tags are selected from a peptide that can be biotinylated, a polyhistidine peptide, or both. In certain implementations, the MHC Class I cSCT protein also includes a secretion signal covalently linked to and N-terminal to the peptide, the secretion signal selected from an HLA secretion signal, an interferon-α2 secretion signal, and an interferon-γ secretion signal.

Of specific interest are HLA protein subunits that include one or more amino acid substitutions selected from H74L, Y84C, Y84A, A139C, D227K, T228A, and A245V MHC Class I cSCT protein. As such, in some implementations, the MHC Class I cSCT protein includes an HLA protein subunit that includes one or more amino acid substitutions selected from the group consisting of H74L, Y84C, Y84A, A139C, D227K, T228A, and A245V.

In general, the peptide is an antigen peptide, a self peptide, or a placeholder peptide. A peptide antigen is a peptide that fits in the binding pocket of an MHC Class I protein complex or an MHC Class I SCT protein and is recognized by CD8+ T cells. In some implementations, the peptide is about 8-14 amino acids long (e.g., 8, 9, 10, 11, 12, 13, 14 amino acids long). However, peptide antigens that are longer or shorter could also be utilized. Examples of the antigen peptide include, but are not limited to, a tumor-associated peptide, a neoantigen peptide, an autoimmune peptide, a fungal peptide, a bacterial peptide, and a viral peptide.

Also provided are nucleic acids encoding the disclosed MHC Class I cSCT proteins. In certain implementations, the nucleic acid molecule also encodes a secretion signal covalently linked to and N-terminal to the peptide, the secretion signal selected from an HLA secretion signal, an interferon-α2 secretion signal, and an interferon-γ secretion signal. In some implementations, the nucleic acid is codon-optimized for mammalian expression, such as for expression in human cells. In many implementations, the nucleic acid molecule is included in a vector, such as a mammalian expression vector. Of particular interest is where the mammalian expression vector is plasmid pcDNA3.1. Also provided are cell lines, such as a human cell lines, transformed with a nucleic acid molecule and/or vector of the disclosure. Examples of the cell line include an HEK293 cell line, such as the Expi293F™ cell line.

### II. MHC Class I Soluble Single Chain Dimers

The MHC Class I sSCD proteins of the disclosure are produced by cleavage of the cSCT protein using a protease that cleaves at the unique protease cleavage site. Briefly, cleavage frees the peptide from the cleavable SCT protein, which produces the soluble dimer. The soluble dimer protein may then be replaced or otherwise loaded with a different peptide of interest. Purification can by carried out by various techniques, and preferably by including one or more purification tags with MHC Class I sSCD protein. Purification tags also find use for attaching the proteins to a surface for reconstitution, screening and the like. The cSCT and sSCD proteins are generally soluble in aqueous solutions and stable, making handling and storage more robust. The MHC Class I sSCD can be produced free of the peptide, as well as reconstituted with the same or different peptide for a given end use. For example, of particular interest is where the sSCD protein-peptide complex is assembled as a stable multimer, such as a tetramer, and applied to a T cell population.

As such, the MHC Class I sSCD protein includes as covalently linked first and second subunits in the N-terminal to C-terminal direction: a β2m protein subunit and a HLA protein subunit. In many implementations, the sSCD protein includes a third subunit that is non-covalently bound by the β2m protein and HLA protein subunits, the third subunit including a peptide of interest, such as a peptide selected from an antigen peptide, a self peptide, or a placeholder peptide. Examples of antigen peptides, include, but are not limited to, tumor-associated peptides, neoantigen peptides, autoimmune peptides, fungal peptides, bacterial peptides, and viral peptides. Examples of HLA proteins include, but are not limited to, HLA proteins having one or more amino acid substitutions selected from H74L, Y84C, Y84A, A139C, D227K, T228A, and A245V. Libraries of the MHC Class I sSCD proteins loaded with a plurality of different peptides are also provided.

### III. Identifying Antigen-Specific CD8+ T Cells

In certain implementations, the disclosure provides a method of identifying an antigen-specific CD8+ T cell. This method includes contacting a T cell population with one or more stable multimers of a MHC Class I sSCD protein; and identifying a CD8+ T cell reactive thereto. In as many implementations, the method further includes sequencing the T cell receptor (TCR) of the identified antigen-specific CD8+ T cell; and producing a population of T cells expressing the antigen-specific TCR. In some implementations, the method further includes administering the population of T cells expressing the antigen-specific TCR to a subject in need thereof. Of particular interest is where the subject has cancer and the antigen-specific TCR is reactive to an antigen from a tumor sample obtained from the subject.

### EXAMPLES

The following examples are provided to illustrate certain particular features and/or aspects of the disclosure. These examples should not be construed to limit the disclosure to the particular features or aspects described.

### Example 1—Production of Cleavable MHC Class I Single Chain Trimers

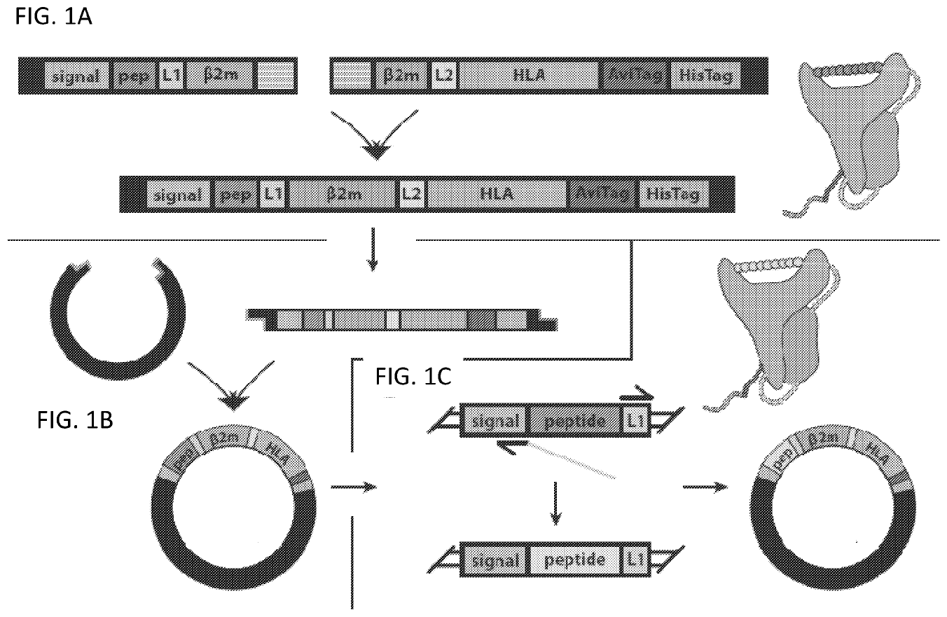

cSCT Template Production: Cleavable Class I SCT (cSCT)-encoded plasmids were constructed using Gibson assembly methods for insertion into a plasmid (such as pcDNA3.1 Zeo (+) plasmid (Thermo Fisher Scientific)) (FIG. 1A). A protease cleavage site (such as a TEV cleavage site) was incorporated into L1. Briefly, the cSCT inserts were designed to be modular to allow for any choice of L1 to be paired with any choice of HLA allele. Because β2m has no allelic variation in the human species, the SCT was split into two Gibson assembly fragments within this region to allow for decoupling of L1 from HLA. Fragments were PCR-amplified with KOD HotStart Hi-Fi polymerase (MilliporeSigma), and joined together by Gibson assembly using NEBuilder HiFi DNA Assembly Master Mix (New England Biolabs). The PCR-amplified Gibson product's flanking regions were digested by EcoRI and XhoI (New England Biolabs) to be ligated into the MCS region of pcDNA3.1 at the same enzyme recognition sites (FIG. 1B). Codon optimization was applied to the designed fragments under three considerations: 1) selection of only highly prevalent codons in the human species, 2) avoidance of continuous gene segments (24+ bp) where GC content is above 60% (to avoid error rates during synthesis), and 3) avoidance of key recognition cut sites within the fragments, which must only exist at the flanks of the Gibson product for insertion into pcDNA vector. The design of the second fragment (encoding HLA allele) was automated with a Python script, encompassing all aforementioned design criteria and accounting for all alleles from Class I HLA-A, B, C loci. The protein sequences of each HLA allele were obtained from an FTP server hosted by The Immuno Polymorphism Database (ftp.ebi.ac.uk/pub/databases/ipd/imgt/hla/fasta/). To date, all existing Class I HLA sequences from the IMGT database have been converted in this manner into ready-to-order DNA sequences.

cSCT Peptide Library Production: A PCR-facilitated approach was implemented to enable high-throughput substitution of peptides into cSCT-encoded plasmids. Briefly, for any given peptide substitution, a peptide-encoded reverse primer (binding to the signal sequence upstream of peptide region) and a forward primer (binding to L1 downstream of peptide region) was required. The peptide-encoded primer varied for any given peptide, while the forward primer remained fixed across all peptide elements (unless one chooses to use a different L1/HLA template plasmid). In this manner, a cSCT plasmid library, encompassing n peptides and m templates, required the purchase of n+m total primers. Extension PCR was conducted with KOD Hot Start polymerase (MilliporeSigma). The product was phosphorylated and ligated with a mixture of T4 Polynucleotide Kinase and T4 DNA Ligase, and then template DNA was digested with DpnI (New England Biolabs). The peptide-substituted plasmids were then transformed into One Shot TOP10 Chemically Competent E. coli (Thermo Fisher Scientific). Plasmids were verified by Sanger sequencing using a Python script prior to use in transfection.

cSCT Expression: Purified cSCT plasmids were transfected into Expi293 cells (Thermo Fisher Scientific) within 24-well (2.5 ml capacity) plates. Briefly, 1.25 μg of plasmid was mixed with 75 μl Opti-MEM reduced serum media. Then 7.5 μl of ExpiFectamine Reagent was mixed with 70 μl Opti-MEM reduced serum media, incubated at room temperature for 5 minutes, and combined with the plasmid mixture. After a 15-minute room temperature incubation, the solution was added to 1.25 ml of Expi293 cells at 3 million cells/ml into a 24-well plate, which was then shaken at 225 RPM at 37° C. in 8% CO2 overnight. Twenty hours later, a solution containing 7.5 μl of ExpiFectamine Transfection Enhancer 1 and 75 μl of ExpiFectamine Transfection Enhancer 2 was added to each well. The plate was kept on the shaker using aforementioned settings for a total of 4 days from start of transfection. The supernatant of the transfection solution was collected and filtered through 0.22 μm PVFD membrane syringe filters (MilliporeSigma) prior to yield analysis via SDS-PAGE. The supernatant solutions of cSCTs which expressed at high yield were concentrated down to 200 μl PBS using 30 kDa centrifugal filter units (Amicon) and subsequently biotinylated with BirA enzyme kit (Avidity) overnight. The biotinylated cSCTs were then purified with HisTag resin tips (Phynexus) and desalted back into PBS buffer with Zeba 7KMWCO spin desalting columns (Thermo Fisher Scientific). For long-term storage, the SCTs were re-suspended into 20% glycerol w/v prior to storage at −20° C.

cSCT Yield Characterization: After 4 days of transfection, a 15 μl solution containing 3:1 mix of transfection supernatant and Laemmli buffer with 10% β-mercaptoethanol was denatured at 100° C. for 10 minutes, and subsequently loaded into Bio-Rad Stain-Free gels for SDS-PAGE (200V, 30 minutes). Images were obtained using a Bio-Rad ChemiDoc MPgel imaging system (manual settings: 45 seconds UV activation, 0.5 second exposure). To identify a consistent approach for analyzing cSCT expression, a custom Python script was developed specifically for the analysis of cSCT proteins run on Stain-Free gels (Bio-Rad). The script allowed for user-defined selection of protein bands of interest, and provided background reduction and uniform normalization of SCT yield across all gels given the consistent use of a control protein lane. The accuracy of this approach was measured by SDS-PAGE of titrated, pre-quantified samples of purified cSCTs to demonstrate a 99% correlation between true protein A280 concentration (as measured by NanoDrop 8000 Spectrophotometer) and quantified relative band intensity. cSCTs which expressed above an established cutoff for yield were selected for subsequent biotinylation and purification steps.

Thermal Stability Characterization: SYPRO™ Orange Protein Gel Stain was purchased from ThermoFisher Scientific and diluted with H₂O to give a 100× working solution. To each 19 μl aliquot of Class I cSCT protein solution (diluted to 10 μM, if possible), 1 μl of the 100× dye solution was added. A Bio-Rad thermal cycler equipped with a CFX96 real-time PCR detection system was used in combination with Precision Melt Analysis software to obtain melting curves of each SCT sample. Thermal ramp settings were 25° C. to 95° C., 0.2° C. per 30 seconds.

Peptide Stimulation: The thawed PBMCs were incubated in complete R10 media (500 ml of RPMI 1640; 50 mL Heat-inactivated FBS; 5 ml of Pen/strep (100 U/mL penicillin and 100 μg/mL streptomycin); 1× GlutaMAX) by adding 1 μM of peptide and anti-CD40 antibody (1 μg/mL) for 16 hrs. On the next day, the PBMCs were washed and stained with Annexin V-BV421 (1 μg/mL), CD8-FITC antibody (1 μg/mL) and CD137-PE antibody (1 μg/mL) for 10 mins at 4° C. Activation-induced expression of CD137 by peptide stimulation permitted the sorting of antigen specific T-cells into tubes using FACS sorter equipment.

### Example 2—Production of cSCTs

We were initially interested in seeing whether two HLA mutations (Y84C and A139C) could be used in the context of SCTs to provide additional stability to the MHC and therefore improve expression yield or protein function. Inclusion of these mutations (D9 template, a.k.a. DS-SCT) in the presence of (GGGGS) 3 (SEQ ID NO: 18) L1 linker for the WT1 peptide resulted in the best tetramer binding performance against the C4 TCR (data not shown).

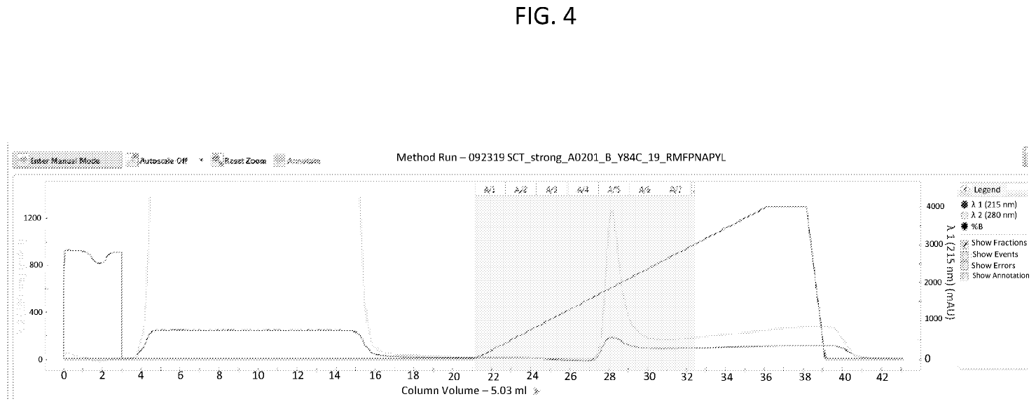

We then used this DS-SCT template to introduce a TEV protease cleavage site into the L1 linker. As previously described, Moritz et al. (supra) were able to produce a large amount of the refolded pMHC using bacterial expression, but ideally, one would aim to study a human-origin protein utilizing a mammalian derived post-translational modifications, especially when the protein expression system has the added capability of generating already folded product in high yield. The DS-SCTs we generated fulfill these criteria, but require the presence of a peptide upstream of L1 to stabilize the construct before the SCT can be secreted into media. We took advantage of this requirement to maximize protein yield of the DS-SCT by selecting a peptide which generally gives high yield (MART-1). Then, to create a peptide-free DS-SCD (single-chain dimer), we introduced the TEV protease cleavage site into the L1 linker upstream of each repeating GGGGS (SEQ ID NO: 1) unit (FIG. 2 and FIG. 3). These MART-1-loaded TEV DS-SCTs (termed cleavable SCTs, or cSCTs) have been shown to express at relatively similar yields to the unmodified MART-1 DS-SCT. We have also shown that SCTs can easily be transfected at large volumes and purified for larger yields via size-exclusion FPLC (FIG. 4).

### Example 3—Functional Testing of cSCTs

Having demonstrated expression of cSCTs, functionality was demonstrated by peptide cleavage and elution, peptide binding, stability assays, and functional binding, as outlined below:
Linker cleavage: TEV protease and elution steps were performed to remove the MART-1 peptide from the cSCT to generate a SCD. This construct was designed to remain stable due to the dithiol mutations introduced into the HLA chain (Y84C and A139C), which has been previously reported for native MHCs.
Peptide binding: peptides of interest were subsequently loaded into the peptide-free SCDs via simple incubation methods, similar to those used for UV exchange protocols.
Stability assay: Functional assays and/or ELISA assays making use of anti-β2m antibodies were used to assess the stability of peptide-bound SCDs.
Functional binding: The peptide-bound SCDs were tetramerized and used to bind against a TCR-transduced T cell line.

Various cSCTs reagents prepared as above were treated with TEV protease and loaded with antigen peptides of interest. Enzyme cleaved cSCTs template (without removal of peptide) was stable for peptide exchange and functional for staining. The cSCTs reagents were also stable either in the form of cleaved template (ready-to-change with new peptide) or as peptide-exchanged tetramers. In order to assess purity of the SCDs after undergoing MART-1 cleavage, purification, and target peptide loading, MART-1 TCR-transduced Jurkat cells were admixed to assess the relative proportions of tetramer-bound MART-1 Jurkat cells versus tetramer-bound target peptide-specific T cells.

There were several advantages observed for the cSCT and SCD proteins as produced and tested. For example, SCD tetramer loaded with a given peptide antigen exhibited similar specific binding for multiple SCD template designs. Moreover, treating cSCTs templates with TEV protease followed immediate incubation with a different peptide allowed characterization and thus selection of a desired place holder peptide. As such, different placeholder peptides can be used to balance the desired pMHC tetramer structure stability with peptide exchange (e.g., stronger place holder peptide binder can benefit stability as well as exchange). Another advantage was that the protease cleaved cSCT template with or without peptide exchange remained stable and functional.

In addition, TCR-SCD tetramer binding was found to be highly epitope restricted and capable of discriminating single amino acid differences between otherwise identical antigen peptides. Conversely, the SCD template generated low non-specific binding for un-matched epitopes thereby limiting false positive results. Compared to covalently linked SCTs, the results indicate that the SCD proteins may allow the peptides to be presented more naturally.

The results further indicate that a single or library of protease cleaved cSCT templates can be tested with multiple different peptides of interest from multiple different sources (e.g., peptides can be produced by recombinant, chemical, or a combination thereof, and used for exchange). For example, combinatorial peptide libraries from different sources could be interrogated in a multiplex fashion with deconvolution carried out by various techniques such as mass spectrometry.

The results further indicate that the peptide-free SCD approach may provide additional insight into the mechanism of peptide loading and presentation. As outlined by Anjanappa et al., peptide-free refolded A*02:01 MHC was needed to study how the A*02:01 peptide-binding groove reacts to the presence of a peptide (Anjanappa et al., “Structures of peptide-free and partially loaded MHC class I molecules reveal mechanisms of peptide selection.” Nature Communications, 11 (1): 1314 December 2020). Prior to this work, no crystal structure of a peptide-free MHC was available; the DS mutation introduced to stabilize MHCs therefore should allow for a peptide-free MHC construct to be crystallized, providing insight into peptide-binding dynamics. The introduction of the TEV linker into the high throughput platform also demonstrated that SCDs can be generated on a larger scale across multiple HLA alleles, therefore allowing for the same type of crystallography and molecular dynamics simulation work to be applied across other highly prevalent HLA alleles.

### Example 4—Production and Analysis of Single-Chain Dimer pMHCs

### Methods

Cleavable SCT template was treated with TEV protease for 1 hour to remove the covalent linkage between contemporary peptide (MART1 peptide for A*02:01, or EBV peptide for A*11:01). Peptide-receptive SCD reagents were concentrated by removal of unbonded peptides and TEV protease using size-exclusive ultra-centrifugation. Peptide-receptive SCD reagents were incubated with the desired peptide at 32° C. for 30 minutes to maximize the peptide exchange efficiency. Peptide-loaded SCD reagents were multimerized onto fluorochrome-labeled streptavidin. Alternatively, TEV protease treated peptide-receptive SCD reagents were stored in PBS at 4° C. for future use.

### Results

### Efficient Antigen-Specific T Cell Detection Using Peptide-Receptive SCD Reagents

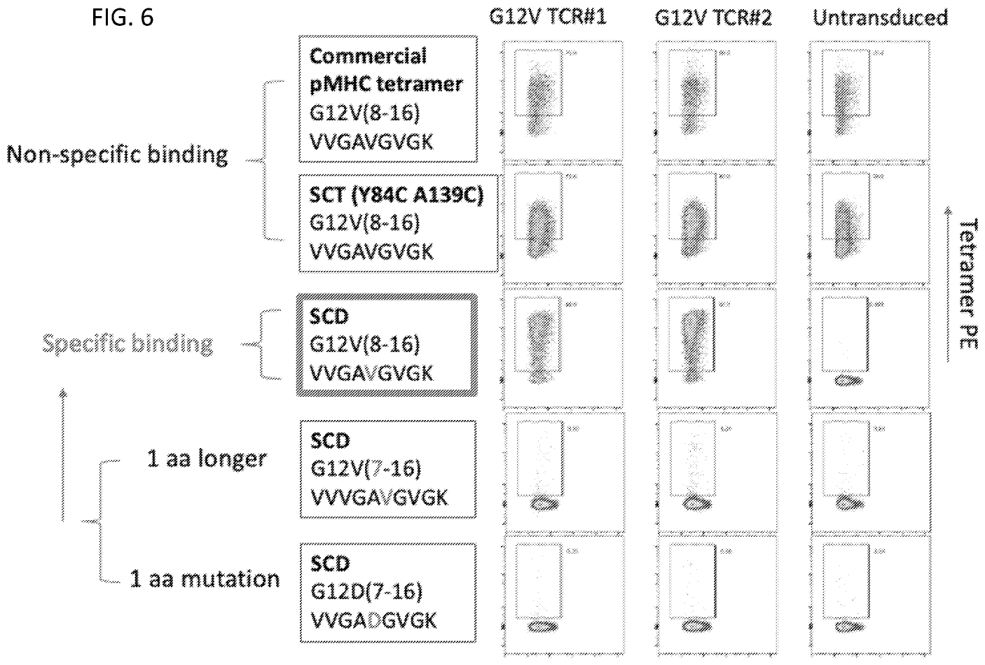

To investigate whether the peptide-loaded SCD reagents is functional in the detection of antigen-specific T cells, we prepared peptide-loaded SCD reagents for HLA-A*11:01 using following peptides: G12V (8-16)_VVGAVGVGK (SEQ ID NO: 7), G12V (7-16)_VVVGAVGVGK (SEQ ID NO: 8), G12D (8-16)_VVGADGVGK (SEQ ID NO: 9). To compare the performance of G12V-SCD reagents with G12V-SCT reagents, we prepared the SCT reagents encoding the same peptide G12V (8-16)_VVGAVGVGK (SEQ ID NO: 7). Flow cytometry analysis on T cell lines expressing G12V-specific TCRs is shown in FIG. 6. Both SCT tetramer and commercial pMHC tetramer for A*11:01 G12V (8-16)_VVGAVGVGK (SEQ ID NO: 7) showed significant non-specific binding on untransduced cells. In contrast, SCD tetramer loaded with the same G12V (8-16)_VVGAVGVGK (SEQ ID NO: 7) peptide showed high binding signal against cognate TCRs (TCR #1 and TCR #2) with ultralow non-specific binding against untransduced cell line. Moreover, SCD tetramer loaded with G12V (7-16)_VVVGAVGVGK (SEQ ID NO: 8), containing one extra amino acid than G12V (8-16), did not bind to TCR #1 or TCR #2. SCD tetramer loaded with G12D (8-16)_VVGADGVGK (SEQ ID NO: 9), containing one altered amino acid than the cognate G12V (8-16) peptide, also did not bind to either TCR #1 or TCR #2. Collectively, these results indicate that potentially altered binding motifs between tested TCRs and similar peptides can be distinguished via sensitive staining using SCD reagents. In conclusion, SCD reagents demonstrated improved specificity against cognate TCRs comparing to SCT tetramer and commercial pMHC tetramer in some cases.

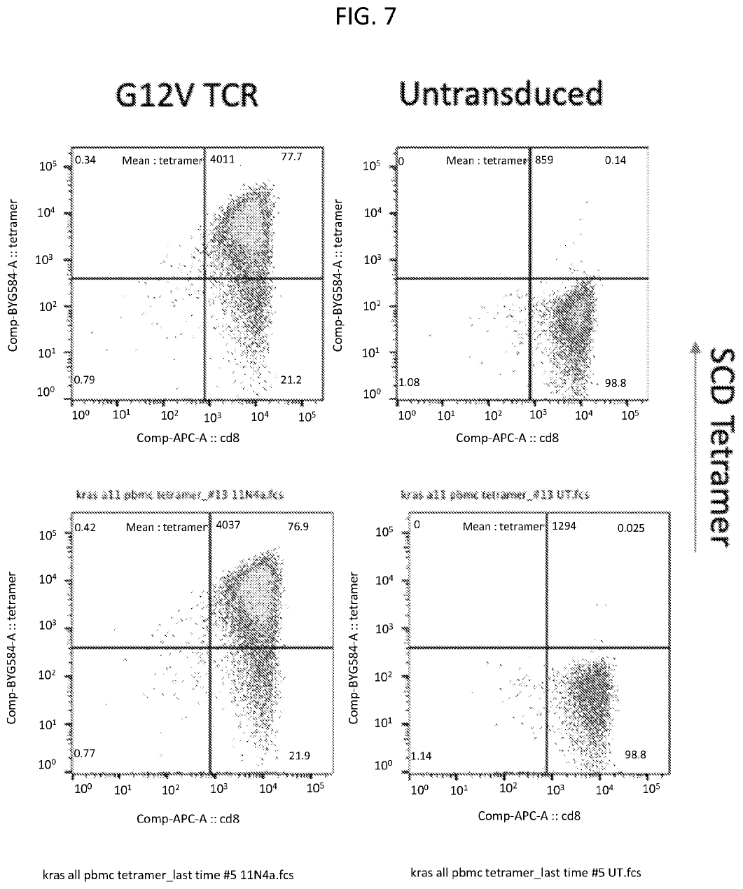

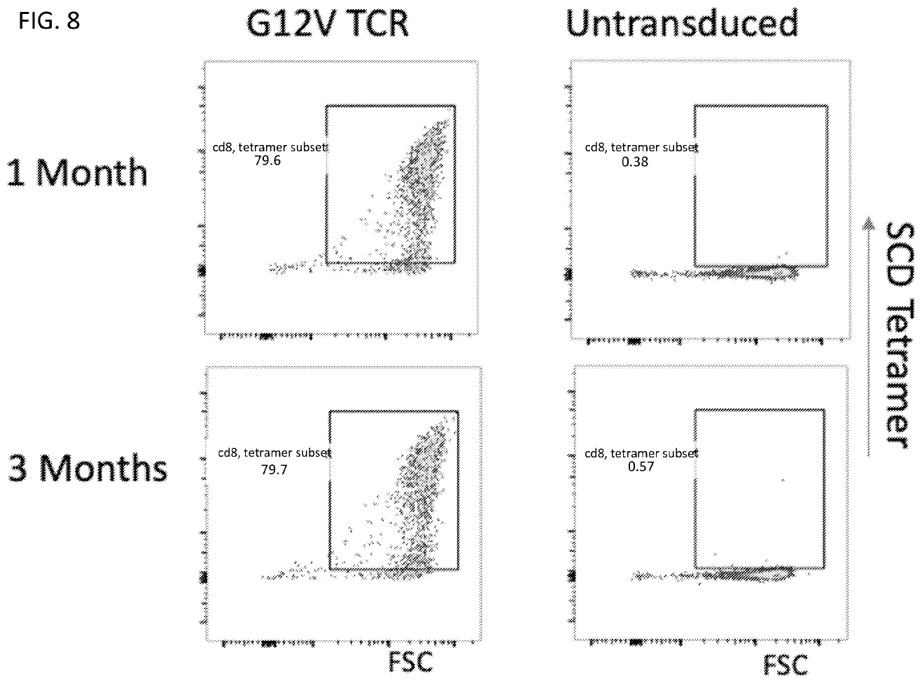

We investigated the stability of SCD reagents in the format of ready-to-use peptide-loaded multimer, or as a peptide-receptive monomer. The purified SCD template can be stored in PBS containing 20% glycerol at −20° C. for years similar to SCT reagents. SCD template can be treated with TEV protease and store in PBS at 4° C. as a peptide-receptive monomer. This peptide-receptive format of SCD reagent can be directly used for desired peptide loading, and multimerization. Alternatively, the peptide-loaded and tetramerized SCD reagent can also be stored in PBS at 4° C. for future use of cell staining. The stability of SCD reagents in two conditions is shown in FIG. 7. In condition A (top), SCD template was treated with TEV protease, stored in PBS at 4° C. for two weeks, then incubated with G12V (8-16) peptide, incubated with tetramer-PE, then stained cells. In condition B (bottom), SCD template was treated with TEV protease, immediately incubated with G12V (8-16) peptide, incubated with tetramer-PE, stored at 4° C. for two weeks before staining cells. SCD reagents from both condition A and condition B showed uncompromised tetramer binding signals against the cognate TCR after 2 weeks storage. FIG. 8 shows that the TEV protease treated, peptide-receptive SCD monomer was stable for up to 3 months and showed similar staining performance comparing to the reagent post one month of storage. In conclusion, this demonstrates that SCD reagent is stable for at least two weeks as ready-to-use peptide-loaded tetramer, and is stable for at least 3 months as peptide-receptive SCD monomer.

It will be apparent that the precise details of the methods or compositions described may be varied or modified without departing from the spirit of the described aspects of the disclosure. We claim all such modifications and variations that fall within the scope and spirit of the claims below.

1. A major histocompatibility complex (MHC) Class I cleavable single chain trimer (cSCT) protein, the cSCT protein comprising as covalently linked subunits in the following N-terminal to C-terminal order a peptide, a unique protease cleavage site, a β2 microglobulin (β2m) protein, and a human leukocyte antigen (HLA) protein.

2. The MHC Class I cSCT protein of claim 1, wherein the unique protease cleavage site is comprised as a linker joining the peptide to the β2m protein.

3. The MHC Class I cSCT protein of claim 2, wherein the MHC Class I cSCT protein comprises as covalently linked components in the following N-terminal to C-terminal order: the peptide; the unique protease cleavage site-β2m linker (L1); the β2m protein; a β2m-HLA linker (L2); the HLA protein; and optionally, one or more purification tags.

4. The MHC Class I cSCT protein of claim 3, wherein the unique protease cleavage site-β2m linker L1 comprises a Gly-Gly-Gly-Gly-Ser (GGGGS; SEQ ID NO: 1) amino acid repeat, and the unique protease cleavage site is internal to, or flanking, the GGGGS (SEQ ID NO: 1) amino acid repeat.

5. The MHC Class I cSCT of claim 1, wherein the unique protease cleavage site is a tobacco etch virus (TEV) protease cleavage site.

6. The MHC Class I cSCT protein of claim 5, wherein the unique protease cleavage site is a TEV protease cleavage site that recognizes the amino acid sequence Glu-Asn-Leu-Tyr-Phe-Gln-Gly/Ser (ENLYFQG/S; SEQ ID NO: 2) and cleaves between the Gln and Gly/Ser residues, and wherein the unique protease cleavage site-β2m linker L1 is selected from: ENLYFQGGGGSGGGGSGGGGS (SEQ ID NO: 3); GGGGSENLYFQGGGGSGGGGS (SEQ ID NO: 4); GGGGSGGGGSENLYFQGGGGS (SEQ ID NO: 5); and GGGGSGGGGSGGGGSENLYFQG/S (SEQ ID NO: 6).

7. The MHC Class I cSCT protein of claim 3, wherein the MHC Class I cSCT protein comprises one or more purification tags and the one or more purification tags are selected from a peptide that can be biotinylated and a polyhistidine peptide.

8. The MHC Class I cSCT protein of claim 1, wherein the HLA protein subunit comprises one or more amino acid substitutions selected from the group consisting of H74L, Y84C, Y84A, A139C, D227K, T228A, and A245V.

9. The MHC Class I cSCT protein of claim 1, wherein the peptide is an antigen peptide, a self peptide, or a placeholder peptide.

10. The MHC Class I cSCT protein of claim 9, wherein the antigen peptide is selected from a tumor-associated peptide, a neoantigen peptide, an autoimmune peptide, a fungal peptide, a bacterial peptide, and a viral peptide.

11. A nucleic acid molecule encoding a MHC Class I cSCT protein of claim 1.

12. A vector comprising the nucleic acid molecule of claim 11.

13. A cell transformed with the vector of claim 12.

14. A major histocompatibility complex (MHC) Class I soluble single chain dimer (sSCD) protein produced by cleavage of the cSCT protein of claim 1 at the unique protease cleavage site.

15. The MHC Class I sSCD protein of claim 14, wherein the MHC Class I sSCD is free of the peptide.

16. The MHC Class I sSCD protein of claim 14, wherein the MHC Class I sSCD is reconstituted with a peptide selected from an antigen peptide, a self peptide, or a placeholder peptide.

17. The MHC Class I sSCD protein of claim 16, wherein the sSCD protein and peptide is assembled as a stable multimer.

18. The MHC Class I sSCD protein of claim 17, wherein the stable multimer is a tetramer.

19. The MHC Class I sSCD protein of claim 16, wherein the stable multimer is attached to a surface through a purification tag.

20. A major histocompatibility complex (MHC) Class I soluble single chain dimer (sSCD) protein, the sSCD protein comprising as covalently linked first and second subunits in the N-terminal to C-terminal direction a β2 microglobulin (β2m) protein subunit and a human leukocyte antigen (HLA) protein subunit.

21. The MHC Class I sSCD protein of claim 20, wherein the sSCD protein comprises a third subunit that is non-covalently bound by the β2m protein and HLA protein subunits, the third subunit comprising a peptide selected from an antigen peptide, a self peptide, or a placeholder peptide.

22. The MHC Class I sSCD protein of claim 21, wherein the sSCD protein is stable in aqueous solution.

23. The MHC Class I sSCD protein of claim 21, wherein the antigen peptide is selected from a tumor-associated peptide, a neoantigen peptide, an autoimmune peptide, a fungal peptide, a bacterial peptide, and a viral peptide.

24. The MHC Class I sSCD protein of claim 20, wherein the HLA protein comprises one or more amino acid substitutions selected from the group consisting of H74L, Y84C, Y84A, A139C, D227K, T228A, and A245V.

25. The MHC Class I sSCD protein of claim 20, further comprising one or more purification tags.

26. A library comprising a plurality of MHC Class I sSCD protein of claim 21.

27. A method of identifying an antigen-specific CD8+ T cell, comprising:
contacting a T cell population with one or more of the stable multimers of a MHC Class I sSCD protein of claim 17; and
identifying a CD8+ T cell reactive thereto.


SEQUENCE LISTING (19 sequences)

SEQ ID NO: 1 - protein, 5 aa, synthetic construct
GGGGS

SEQ ID NO: 2 - protein, 7 aa, synthetic construct
  VARIANT 7: note = X is G or S
ENLYFQX

SEQ ID NO: 3 - protein, 21 aa, synthetic construct
ENLYFQGGGGSGGGGSGGGGS

SEQ ID NO: 4 - protein, 21 aa, synthetic construct
GGGGSENLYFQGGGGSGGGGS

SEQ ID NO: 5 - protein, 21 aa, synthetic construct
GGGGSGGGGSENLYFQGGGGS

SEQ ID NO: 6 - protein, 22 aa, synthetic construct
  VARIANT 22: note = X is G or S
GGGGSGGGGSGGGGSENLYFQX

SEQ ID NO: 7 - protein, 9 aa, synthetic construct
VVGAVGVGK

SEQ ID NO: 8 - protein, 10 aa, synthetic construct
VVVGAVGVGK

SEQ ID NO: 9 - protein, 9 aa, synthetic construct
VVGADGVGK

SEQ ID NO: 10 - protein, 25 aa, synthetic construct
ELAGIGILTVGGGGSGGGGSGGGGS

SEQ ID NO: 11 - protein, 31 aa, synthetic construct
ELAGIGILTVENLYFQGGGGSGGGGSGGGGS

SEQ ID NO: 12 - protein, 11 aa, synthetic construct
ELAGILGILTV

SEQ ID NO: 13 - protein, 25 aa, synthetic construct
ELAGIGILTVGGGGSGGGGSGGGGS

SEQ ID NO: 14 - protein, 31 aa, synthetic construct; identical to SEQ ID NO: 11

SEQ ID NO: 15 - protein, 31 aa, synthetic construct
ELAGIGILTVGGGGSENLYFQGGGGSGGGGS

SEQ ID NO: 16 - protein, 31 aa, synthetic construct
ELAGIGILTVGGGGSGGGGSENLYFQGGGGS

SEQ ID NO: 17 - protein, 31 aa, synthetic construct
ELAGIGILTVGGGGSGGGGSGGGGSENLYFQ

SEQ ID NO: 18 - protein, 15 aa, synthetic construct
GGGGSGGGGSGGGGS

SEQ ID NO: 19 - protein, 9 aa, synthetic construct
RMFPNAPYL